(19) United States
(12) Patent Application Publication
(10) Pub. No.: US 2022/0386674 A1
(43) Pub. Date: Dec. 8, 2022

(54) USE OF ALPHA-LACTALBUMIN ENRICHED WHEY PROTEIN EXTRACT AS A SOURCE OF CHOLESTEROL

(71) Applicant: SOCIETE DES PRODUITS NESTLE S.A., Vevey (CH)

(72) Inventors: JONATHAN O'REGAN, Ithaca, NY (US); CIAN TERENCE MOLONEY, Linwood, NJ (US); DEBORAH O'CONNOR, Farranshone (IR)

(21) Appl. No.: 17/755,939

(22) PCT Filed: Nov. 9, 2020
(86) PCT No.: PCT/EP2020/081445

§ 371 Date: May 12, 2022

(30) Foreign Application Priority Data
Nov. 13, 2019 (EP) 19208776.5

(51) Int. Cl. A23L 33/19; A23J 3/08; A23L 33/00; A61K 38/38

(52) U.S. Cl. CPC A23L 33/19; A23J 3/08; A23L 33/40; A61K 38/38; A23V 2002/00

(57) ABSTRACT

The present invention relates to use of an α-lactalbumin enriched whey protein extract as a source of cholesterol in a synthetic nutritional composition for an infant or child wherein, the α-lactalbumin enriched (WPE) is obtained by a process comprising steps of (i) acidifying a whey protein product to pH 4 or below; (ii) forming a low calcium whey protein product by concentrating the proteins in the acidified whey protein until the calcium to protein ratio is less than about 0.001 and, (iii) Precipitating α-lactalbumin from the low-calcium whey protein product, wherein said precipitating step includes the sub-steps of: (a) diluting the low-calcium whey protein product, (b) adjusting the pH of the diluted low-calcium whey protein product to between 4 and 5 to form a precipitate and soluble proteins, and (c) Separating the precipitate proteins from the soluble proteins; wherein the WPE is added to said composition in an effective amount sufficient to ensure that said synthetic nutritional composition has a final concentration of cholesterol found in human breast milk.

### FIELD OF THE INVENTION

The present invention relates to the use of α-lactalbumin enriched whey protein extract as a source of cholesterol, and to the use of said α-lactalbumin enriched whey protein extract to optimize/configure the cholesterol concentration in a synthetic nutritional composition for an infant or child such that the cholesterol content resembles the content in human breast milk and ranges from 90 to 150 mg/L of the composition. The invention further relates to the use of said synthetic nutritional composition for an infant or child to support and/or optimise growth and development, immune response, galactose metabolism and cytoskeleton remodeling.

### BACKGROUND TO THE INVENTION

Breast-feeding is recommended for all infants. However, in some cases breast-feeding is insufficient or not possible for medical reasons. In these situations infant formula can be used as a substitute for breast milk. However, studies have shown that the composition of infant formula is not identical to that of breast milk, and that it may not always have identical effects on the body. In light of this, and in light of the fact that breast milk is considered the gold standard when it comes to infant nutrition, a goal of infant formula manufacturers is to further develop the compositions of their infant formulas and growing-up milks and to bring them closer to breast milk.

Human milk is the ideal source of nutrition for infants, and is broadly composed of water, carbohydrates, lipids, proteins, vitamins and minerals. The lipid fraction of human milk occurs in various forms and is responsible for a wide variety of functions. The majority of lipids are in the form of neutral triglycerides (98%) that typically provide greater than 50% of the caloric intake of the newborn infant. Cholesterol and polar lipids (PLs)—primarily phospholipids and sphingolipids—make up a relatively small proportion of the lipids (0.2-2.0%) and are found mostly as part of the milk fat globule membrane (MFGM) (Jensen, 1999).

Cholesterol is important for neuron and nervous system development, vitamin D synthesis and functions as a precursor of some bile acids (Beggio, Cruz-Hernandez, Golay, Lee & Giuffrida, 2018; Ohlsson, 2010). Human milk typically contains ˜90-150 mg/L cholesterol and it has been suggested that breast-feeding may be associated with lower total blood cholesterol concentrations later in life compared with formula feeding (Beggio et al., 2018; Wong, Hachey, Insull, Opekun & Klein, 1993). Bovine milk is richer in cholesterol (˜260 mg/L) than human milk, but infant formula typically contains low levels of cholesterol (<5 mg/L) as it is usually formulated with vegetable oils rather than animal fats (Hageman, Danielsen, Nieuwenhuizen, Feitsma, & Dalsgaard, 2019; Claumarchirant, Matencio, Sanchez-Siles, Alegria & Lagarda, 2015; Precht, 2001). Therefore, formula-fed infants have a reduced dietary cholesterol intake and have lower plasma cholesterol levels than breastfed infants (Wong et al., 1993).

The present work describes the development nutritional formulations using an α-lactalbumin-enriched fraction at specific levels to be enriched in cholesterol such that formulations at the time of administration can meet the cholesterol levels of human breast milk (90 to 150 mg/1).

Surprisingly, the inventors have now found that a whey protein extract that has been processed to enrich the α-lactalbumin content by use of a particular process detailed herein comprises cholesterol in a concentration far higher than that found in other whey protein extracts using other methods or skimmed milk powder.

This advantageously enables WPE be used as a source of cholesterol and to optimize/configure the concentration of cholesterol in a composition for an infant or child, while at the same time also providing a source of α-lactalbumin. In the absence of an ingredient having both an enriched content of cholesterol and of α-lactalbumin, it would be required to add two different ingredients in order to add these two components into an infant formula. As cholesterol and α-lactalbumin are typically provided in the form of ingredients comprising significant amounts of additional milk protein, such as whey, the protein content in the infant formula in which two separate ingredients have been added would have a significantly higher protein content compared to a formula in which the present α-lactalbumin enriched whey protein extract is used. Thus, the present α-lactalbumin enriched whey protein extract (WPE) is particularly useful in infant formula intended to have a low protein content.

### SUMMARY OF THE INVENTION

In a first aspect, the present invention relates to use of an α-lactalbumin enriched as a source of cholesterol in a synthetic nutritional composition for an infant or child wherein, said α-lactalbumin enriched (WPE) is obtained by a process comprising:
a. acidifying a whey protein product to pH 4 or below for example a pH in the rage 3.3 to 3.8 such as pH 3.5,
b. forming a low calcium whey protein product by concentrating the proteins in the acidified whey protein until the calcium to protein ratio is less than about 0.001 and
c. precipitating α-lactalbumin from the low-calcium whey protein product, wherein said precipitating step includes the sub-steps of:
   I. diluting the low-calcium whey protein product,
   II. adjusting the pH of the diluted low-calcium whey protein product to between 4 and 5 to form a precipitate and soluble proteins, and
   III. Separating the precipitate proteins from the soluble proteins for example by ultrafiltration e.g. through a 5K-50K molecular weight cut off membrane, and/or by diafiltration.

In step a, the whey protein product may be acidified by the addition of an acid. The acid may be a food grade acid e.g. hydrochloric acid, phosphoric acid, citric acid, and/or sulfuric acid. If a whey protein powder is used as the whey protein product, this must be brought into solution prior to step a.

In one embodiment the step b) is performed for example by ultrafiltration e.g. through a 10K-100K molecular weight cut off membrane, and/or diafiltration.

The advantage of the present invention is using WPE, we can effectively limit the amount of protein in the synthetic nutritional composition such said composition is closer to human breast milk. Addition of other known dairy sources of cholesterol leads an undesirable increase in protein and energy requirements in a synthetic nutritional composition.

In one embodiment, the synthetic nutritional composition, in addition to WPE comprising cholesterol further comprises a2-β-casein.

In one embodiment, the synthetic nutritional composition, in addition to WPE comprising cholesterol further comprises sn-2 palmitate.

In one embodiment, the synthetic nutritional composition, in addition to WPE comprising cholesterol further comprises lactoferrin.

### DETAILED DESCRIPTION

A “α-lactalbumin enriched (WPE)” is herein defined as a whey protein extract that has been processed to increase the amount of α-lactalbumin in the whey protein extract compared to the un-processed whey protein extract.

The WPE obtained by the specific process described herein has been found to be surprisingly rich in cholesterol apart from α-lactalbumin and may advantageously be used to optimise the cholesterol concentration in a synthetic nutritional composition for an infant or child. The WPE may be added to a synthetic nutritional composition in an effective amount, sufficient to ensure that the said composition has a final concentration of cholesterol in a range found in human breast milk.

The invention also provides a synthetic nutritional composition comprising WPE, obtained as detailed herein, in a concentration of 0.8 to 8 g/L. A composition may for example comprise 4 to 5 g/L of WPE and may be formulated for an infant of 6 to 12 months of age, or may be formulated for an child of 12 to 36 months of age. The cholesterol concentration in said compositions may be at least 90 mg/L of the total reconstituted infant formula.

The synthetic nutritional composition for an infant or child may be a composition for consumption by infants either alone or in combination with human breast milk, and may be an infant formula or human breast milk fortifier.

The WPE obtained as described herein (Lac B), or a synthetic nutritional composition comprising it, may be used to provide an infant or child with an optimized amount of cholesterol, it may also be used to support and/or optimise the growth and/or development of an infant or child, to support and/or optimise the immune response of an infant or child, in the treatment and/or prevention of sub-optimal immune response of an infant or child, in the treatment and/or prevention of sub-optimal growth of an infant or child.

It is particularly useful to have cholesterol provided as part as WPE as described herein, because both α-lactalbumin and cholesterol may be used to support and/or optimise the growth of an infant or child, or in the treatment or prevention of sub-optimal growth of an infant or child.

The whey protein product may be a whey protein concentrate prepared in any conventional way from mammalian whey (sweet or acid whey) for example cow, goat, sheep, buffalo, water buffalo, yak, human, camel and/or lama. A whey protein concentrate may for example be obtained from skimmed and/or clarified bovine whey that has been concentrated and/or desalted by common means, e.g. by ultrafiltration (the whey protein product may be the retentate) and/or diafiltration.

A process for obtaining an α-lactalbumin enriched WPE for use in the invention detailed herein is described in U.S. Pat. No. 6,312,755, the contents of which is hereby incorporated by reference.

Accordingly in an embodiment of the present invention the WPE is rich in cholesterol and comprises at least 4 g, preferably at least 4.5 g, more preferably at least 5 g, even more preferably at least 5.5 g and most preferably 5.7 g of cholesterol per 100 g. In a preferred aspect of the invention, the amount of cholesterol is determined in accordance with the method described in Example 1 below.

The term “cholesterol” as used herein preferably refers to a lipid structure consisting of four fused hydrocarbon rings, a hydrocarbon tail and a hydroxyl group, which is an important structural component of animal cell membranes.

In another aspect, the present invention relates to the use of an α-lactalbumin enriched WPE to optimise the cholesterol concentration of a synthetic nutritional composition for an infant or child wherein said α-lactalbumin enriched WPE is obtained by a process as detailed herein.

The α-lactalbumin enriched WPE may be added to a synthetic nutritional composition in any amount effective (an effective amount) to optimise the concentration of cholesterol in said synthetic nutritional composition for an infant or child.

The term “A2 milk” generally refers to a variety of cow's milk that mostly lacks a form of β-casein proteins called A1, and instead has mostly the A2 form. A2 cow's milk is commercially available from the a2 Milk Company (Auckland, New Zealand). Non-cow milk, including that of humans, sheep, goats, donkeys, yaks, camels, buffalo, and others, also contain mostly A2 β-casein, and so the term “A2 milk” is also used in that context.

Given that human breast milk is the gold standard when it comes to infant and/or child nutrition, the concentration of cholesterol in a synthetic nutritional composition for an infant or child may be considered optimised if the concentration of cholesterol is within a range, or above a range, found in human breast milk. Cholesterol is found in animal products, such as meat and poultry, but these are not suitable for inclusion in infant formula. Accordingly, optimisation of the cholesterol level of a synthetic nutritional composition can be most practically achieved through the inclusion of cholesterol-rich dairy materials.

A commercially available composition sold by Lipid Nutrition is Betapol™ B-55, which is a triglyceride mixture derived from vegetable oil in which at least 54% of the palmitic acid is in the sn-2 position of the glycerol molecule. In one embodiment, the fat content of the formula of this invention is about 40-55% Betapol™ B-55 by weight, for example from about 45% to about 50% by weight. Those skilled in the art will appreciate that the percentage of the high sn-2 fat used and the total amount of sn-2 palmitate in the formula may vary, and that a different high sn-2 palmitate oil may be used, without departing from the spirit and scope of the invention.

Cholesterol has been found to be present in breast milk in a concentration range of about 90 to 150 mg/L. Accordingly, an effective amount of the WPE may be an amount sufficient to provide cholesterol in one or more of these ranges or in a higher concentration. An effective amount may also be an amount sufficient to ensure that the synthetic nutritional composition has a final concentration of cholesterol in one or more of these ranges or in a higher concentration when considering other ingredients comprised in the composition that comprise cholesterol e.g. dairy ingredients such as skimmed milk powder and whey protein. Said ingredients may comprise cholesterol innately.

When designing the amount of cholesterol to be in the synthetic nutritional composition for an infant or child, it is necessary to do so with consideration for the overall composition, so that the cholesterol-rich material does not otherwise impact the nutritional composition adversely; for example by introducing undesirable exogenous components. It is well within the purview of the person skilled in the art to determine an effective amount of WPE, to be added to the synthetic nutritional composition for an infant or child, based upon the amount of cholesterol found in human breast milk and the concentration of cholesterol in the WPE, and when applicable the concentration of cholesterol coming from other ingredients comprised in the synthetic nutritional composition for an infant or child.

The optimised concentration of cholesterol may be the concentration of cholesterol in the synthetic nutritional composition upon reconstitution for example with milk or water. It is well within the purview of the skilled person to determine an effective amount taking into consideration the concentration of cholesterol in the α-lactalbumin enriched WPE and when appropriate reconstitution instructions for the synthetic nutritional composition.

A particular advantage of the WPE used in the invention is that it can provide an optimised amount of cholesterol to a synthetic nutritional composition for an infant or child and negate the need to add additional ingredients for this purpose, for example additional ingredients whose addition would be for the sole or primary purpose of increasing the cholesterol concentration e.g. isolated cholesterol. Accordingly, in an embodiment the α-lactalbumin enriched WPE is not used in combination with an additional ingredient whose sole or primary purpose would be to increase the cholesterol concentration in the synthetic nutritional composition, for example it is not used in combination with isolated cholesterol as the levels achieved through the use of WPE are already within the target range.

The term “infant” as used herein refers to a human infant of up to 12 months of age and includes preterm and very preterm born infants, infants having a low birth weight i.e. a new born having a body weight below 2500 g (5.5 pounds) either because of preterm birth or restricted fetal growth, and infants born small for gestational age (SGA) i.e. babies with birth weights below the 10th percentile for babies of the same gestational age.

The term “child” as used herein refers to a human of 1 to 18 years of age, for example a human of 1 to 8 years of age, a human of 1 to 3 years of age, and/or a human of 1 to 2 years of age.

A “preterm” or “premature” means an infant or young child that was not born at term. Generally it refers to an infant born prior to the completion of 37 weeks of gestation.

The WPE formed by the process described herein also serves as a source of protein for example α-lactalbumin, β-lactoglobulin, lactoferrin and immunoglobulins.

The WPE obtained by this process set out hereinabove comprises α-lactalbumin in a concentration in the range of about 28% to 40% of the total protein, for example about 28% to 36% of total protein, and β-lactoglobulin in a concentration in the range of about 8% to about 33% of the total protein, for example 10% to about 29% of total protein. The percentage of α-lactalbumin in the WPE is greater than the percentage of β-lactoglobulin, for example the β-lactoglobulin concentration is not greater than the percentage of the α-lactalbumin content minus 7%.

All percentages disclosed herein with respect to the α-lactalbumin enriched WPE are on a w/w basis unless stated otherwise.

The term “optimised concentration of α-lactalbumin” as used herein refers to a concentration of α-lactalbumin that is within a range found in human breast milk (1.6-3.8 g/L). The α-lactalbumin enriched WPE may be considered to optimise the concentration of α-lactalbumin in a synthetic nutritional composition if the α-lactalbumin concentration for the synthetic nutritional composition is within the range found in human breast milk, when considering other ingredients comprised in the composition that comprise α-lactalbumin e.g. dairy ingredients such as skimmed milk powder and whey protein. Said ingredients may comprise α-lactalbumin innately.

In an embodiment the synthetic nutritional composition comprises the WPE in a concentration within a range of 0.8 to 10 g/L for example 0.8 to 8, 0.8 to 5 g/L, 0.85 to 4.5 g/L, 3 to 4.5 g/L.

In an embodiment at least 10% of the total cholesterol in the synthetic nutritional composition comes from the WPE for example 10 to 100%, 49% to 100%, 49% to 70%.

The synthetic nutritional composition comprises cholesterol in a concentration in a range as in human breast milk or in a higher concentration. Accordingly, the synthetic nutritional composition may comprise cholesterol in a concentration of 10 mg/L or more, for example from 10 to 400 mg/L, or from 10 to 350 mg/L.

In another embodiment of the present invention the composition also comprises α-lactalbumin in a concentration within a range found in human breast milk for example in a range of 1.6 to 3.8 g/L, for example 1.7 to 3 g/L.

A goal of infant formula manufacturers is to mimic the composition of human breast milk. However, the composition of human breast milk is extremely dynamic and changes over time. For this reason synthetic nutritional compositions for infants or children are usually stage based with a particular stage being suitable for use in infants or children falling within a particular age range e.g. stage 1 may be aimed at infants of 0 to 6 months, stage 2 may be aimed at infants of 6 months to 12 months, stage 3 may be aimed at children of 12 to 36 months, stage 4 may be aimed at children of 3 to 8 years. Each stage is formulated so that its composition is considered nutritionally sound with respect to the age range of the infant or child to whom it is directed.

In an embodiment of the present invention there is provided a synthetic nutritional composition for an infant or child comprising 9 to 10 g/L of the α-lactalbumin enriched WPE used in the invention for example 9.5-10.2 g/L. In an embodiment said composition is formulated for an infant of 0 to 12 months. In a more specific embodiment the total concentration of cholesterol in said composition is at least 90 mg/L and more specifically in a range of 90 to 150 mg/L.

In a preferred embodiment, the synthetic nutritional composition is has a low total protein content. The amount of protein can be as low as adequate for the type of composition and the individual intended to consume it, such as for example according to nutritional requirements and/or regulations. For example the protein content is of at most 20 g/L, preferably at most 18 g/L, more preferably at most 16 g/L, even more preferably at most 15 g/L, even more preferable 14 g/L, even more preferably between 5 and 20 g/L, even more preferably 8 to 18 g/L, even more preferably 10 to 16 g/L, more preferably 11 to 15 g/L, more preferably 12 to 14 g/L, such as for example 13.5 g/L.

The synthetic nutritional composition for an infant or child can also comprise any other ingredients or excipients known to be employed in the type of synthetic nutritional composition in question e.g. infant formula.

Non limiting examples of such ingredients include: other proteins, amino acids, carbohydrates, oligosaccharides, lipids, prebiotics or probiotics, essential fatty acids, nucleotides, nucleosides, vitamins, minerals and other micronutrients.

Other suitable and desirable ingredients of synthetic nutritional compositions, that may be employed in the synthetic nutritional compositions for infants or children are described in guidelines issued by the Codex Alimentarius with respect to the type of synthetic nutritional composition in question e.g. Infant formula, growing up milk, HM fortifier, follow on formula, or food stuffs intended for consumption by infants e.g. complementary foods.

The WPE may be added to a synthetic nutritional composition for an infant or child by simply mixing it with other ingredients included in the composition.

Non limiting examples of synthetic nutritional compositions for an infant or child are infant formula, a growing up milk, a composition for infants that is intended to be added or diluted with human breast milk, or a food stuff intended for consumption by an infant and/or child either alone or in combination with human breast milk.

α-lactalbumin is rich in essential and conditionally essential amino acids. Accordingly, the α-lactalbumin enriched WPE of the invention is particularly suitable in low protein synthetic nutritional compositions for infants and children because for a minimal protein intake said infants and children still intake sufficient amino acids to optimise growth and development i.e. be within standard growth curves e.g. WHO standard growth curves.

The WPE used in the present invention and obtained by the process described herein contains a minimum amino acid concentration, in grams per 100 grams of total protein, as follows: arginine 3.1; cystine 1.4; histidine 1.6; isoleucine 1.0; leucine 5.3; lysine 3.9; methionine 0.3; phenylalanine 1.2; threonine 3.2; tryptophan 1.5; tyrosine 0.9; and valine 1.0. Accordingly, it may not be necessary to add amino acids to the synthetic nutritional compositions for infants or children when using this WPE. The non-protein nitrogen content may be about 15% or less of total nitrogen. The total protein content may be between about 12.5% to about 95% for example 35% to 80% or 73% to 77%. The fat content may be about 15% or less. The ash content may be about 4.5% or less.

In an embodiment the synthetic nutritional composition is a low protein infant formula. A low protein infant formula will comprise less than 3.5 g of protein/100 kcal for example less than 2.5 g/100 kcal or less than 2 g/100 kcal. The low protein infant formula may be an infant formula formulated for an infant of up to 12 months of age, for example for an infant of 0 to 6 months of age, or an infant of 6 to 12 months of age.

The synthetic nutritional compositions for infants or children may be prepared by methods well known in the art for preparing the type of synthetic nutritional composition in question e.g. infant formulae, follow on formulae, a composition for infants that is intended to be added or diluted with HM e.g. HM fortifier, or food stuffs intended for consumption by infants either alone or in combination with HM e.g. complementary foods.

An infant formula may for example be prepared by blending appropriate quantities of the α-lactalbumin-enriched whey protein concentrate with skimmed milk, lactose, vegetable oils and fat soluble vitamins in water. These materials may be blended together in quantities sufficient to provide a final concentration of approximately 400 g/L Mineral salts may then be added to the mixture prior to a high temperature/short time pasteurization step. Appropriate mineral salts include calcium chloride, calcium carbonate, sodium citrate, potassium hydroxide, potassium bicarbonate, magnesium chloride, ferrous sulfate, potassium citrate, zinc sulfate, calcium hydroxide, copper sulfate, magnesium sulfate, potassium iodide, sodium selenite, etc. The mixture may then be homogenized and cooled. Heat-labile vitamins and micronutrients may then be added to the mixture. The mixture may then be standardized with deionized water to a final total solids concentration of about 120 to about 135 for example about 129 g/L, which is equivalent to about 670 kcal per litre. The formula may then be sterilized using a conventional ultrahigh temperature or standard retort process. This sterilized material may then be placed in appropriate packaging.

In another aspect of the present invention there is provided the use of the α-lactalbumin enriched whey protein extract obtained as disclosed herein to provide an optimised amount of cholesterol to an infant or child. As disclosed herein, said α-lactalbumin enriched whey protein extract may be added to a synthetic nutritional composition in an amount effective to provide an optimised concentration of cholesterol. The α-lactalbumin enriched whey protein extract obtained as disclosed herein may also provide an optimised amount of α-lactalbumin to an infant or child.

Because human breast milk is the gold standard when it comes to infant nutrition, and because the synthetic nutritional compositions comprising the α-lactalbumin enriched whey protein extract disclosed herein may comprise cholesterol in an optimized concentration, they may be used to provide an optimum amount of cholesterol to an infant and thereby to ensure optimum cholesterol levels in an infant or child.

An optimum cholesterol intake has been associated with support and/or optimization of growth and development; immune response; galactose metabolism; and/or cytoskeleton remodeling in an infant or child.

Accordingly, in another aspect of the present invention there is provided a synthetic nutritional composition for an infant or child for use to treat or prevent sub-optimal growth and development, wherein said synthetic nutritional composition comprises WPE obtained as disclosed herein in an amount effective to provide an optimised concentration of cholesterol. The composition may be a composition described herein.

The synthetic nutritional composition may be for use to prevent sub-optimal growth and development in an infant or child having impaired and/or delayed growth and/or development. It is within the purview of the skilled person to assess whether an infant or child is developing normally or whether the infant or child is suffering from impaired or delayed growth and/or development.

In yet another aspect there is provided the use of a synthetic nutritional composition comprising WPE obtained as disclosed herein in an amount effective to provide an optimised concentration of cholesterol to promote and/or optimise immune response in an infant or child to whom it is administered. The composition may be a composition described herein.

In yet another aspect there is provided the use of a synthetic nutritional composition comprising an α-lactalbumin enriched whey protein extract obtained as disclosed herein in an amount effective to provide an optimised concentration of cholesterol to promote and/or optimise galactose metabolism in an infant or child to whom it is administered. The composition may be a composition described herein.

In yet another aspect there is provided the use of a synthetic nutritional composition comprising an WPE obtained as disclosed herein in an amount effective to provide an optimised concentration of cholesterol to promote and/or optimise cytoskeleton remodeling in an infant or child to whom it is administered. The composition may be a composition described herein.

As disclosed hereinabove, the the synthetic nutritional compositions comprising WPE disclosed herein may also comprise α-lactalbumin in an optimized concentration. Consequently, they may also be used to provide an optimum amount of α-lactalbumin to an infant or child and thereby ensure optimum α-lactalbumin levels in an infant or child.

It should be appreciated that all features of the present invention disclosed herein can be freely combined and that variations and modifications may be made without departing from the scope of the invention as defined in the claims. Furthermore, where known equivalents exist to specific features, such equivalents are incorporated as if specifically referred to in this specification.

There now follows a series of non-limiting examples that serve to illustrate the invention.

### Example 1

Raw Materials & Nutritional Powder Products

Infant Formula Raw Materials

Skimmed milk powder (SMP) samples were obtained from each of four suppliers: one located in Ireland (SMP A; n=5), one in Germany (SMP B; n=5), one in the USA (SMP C; n=5) and one in New Zealand (SMP D; n=5). WPC samples (35% protein) were obtained from each of two suppliers; one located in Ireland (WPC A; n=5) and one in the USA (WPC B; n=5). WPC ingredients enriched in α-lactalbumin was obtained from two sources (i) Lac A; n=2 and (ii) Lac B; n=5. Lac A was produced using a membrane process to selectively retain components larger than α-lactalbumin, thereby enriching the permeate in the target protein. As part of the manufacturing process of Lac B, α-lactalbumin was enriched by acid precipitation at reduced temperature (˜4° C.), followed by a membrane filtration step to enrich the retentate in α-lactalbumin, as well as other relatively large molecules, including fat globules.

2.1.2 Nutritional Formula Powders

Prototype infant formula (IF) or growing up milk (GUM) powders were produced in a pilot plant facility as follows: Dry ingredients (whey source of WPC A and/or Lac B, SNIP, lactose, lipids and soybean lecithin) were added sequentially to reverse-osmosis pre-treated water (˜70° C.) and mixed by agitation until dissolved. The resulting mixtures (˜35% solids) were then homogenised (two-stage homogeniser: 2500 psi and 500 psi for the first and second stages, respectively) heat-treated using a plate heat-exchanger (85° C.×22 sec holding time) and cooled to 10° C. The mixture was then stored for 16 h at 10° C., before the liquids were heated to ˜50° C. and spray dried using a small-scale dryer to produce powders for analysis.

IF prototypes, suitable for infants from birth up to approximately 6 months age, were produced to contain as a whey protein source either Lac B (IF 1; n=5), or WPC A (IF 2; n=2). The whey protein source and SMP quantities were set to target the same protein content (10.4 g/100 g, 60:40 whey:casein ratio), and the lactose content was then varied as necessary to achieve the lactose target (53.0 g/100 g); the ingredients used were otherwise identical in both products and included anhydrous milk fat (45% of blend) and a combination of vegetable oils (55% of blend; as rapeseed, high oleic sunflower, regular sunflower and coconut oils) as lipid sources.

GUM prototypes, suitable for children of approximately one to three years of age, were produced to contain as a whey source either a mixture of Lac B and WPC A (GUM 1; n=5) or only WPC A (GUM 2; n=3). Both GUM products targeted a protein target of 13.7 g/100 g (40:60 whey:casein), and the lactose content was then varied as necessary to achieve the available carbohydrate target (61.0 g/100 g); the ingredients used were otherwise identical in both products and included a combination of vegetable oils (soybean, high oleic sunflower and palm oils) as lipid sources.

Cholesterol Quantification

Cholesterol was quantified by AOAC Official Method 994.10 for measuring cholesterol in food products. Briefly, samples were saponified by refluxing with an ethanol/KOH solution, and the unsaponified fraction containing cholesterol was extracted with high-purity toluene. Cholesterol was derivatised to trimethylsilyl ethers, separated on a cross-linked 5% phenyl-methyl silicone capillary column (0.17 μm film thickness) and quantified by flame-ionisation-detector gas chromatography.

Cholesterol Content of Ingredients

All of the SMP samples analysed had similar cholesterol levels, ranging from 20.7 to 24.2 mg/100 g (Table 1). Assuming liquid skimmed milk to contain 91% water and 9% solids this corresponds with a range of approximately 186-218 mg/L in the liquid product; this is lower than, but reasonably close to, reported levels of ˜260 mg/L for whole bovine milk (Precht, 2001). This suggests that most of the milk cholesterol is retained with other Milk fat globule membrane (MFGM) type components in the skimmed milk phase, rather than being lost in the cream phase during fat skimming.

WPC A and B were higher in cholesterol than the SMP samples, containing mean levels of 77.0 and 85.3 mg/100 g, respectively; this was likely as a result of these materials having higher lipid concentrations than SMP. Although the basic concepts are consistent, not all WPC ingredients are produced necessarily with the same procedure; so as well as potential genetic and feeding differences to give different milk compositions, processing differences may be a further differentiating factor between the WPCs analysed. The mean cholesterol level of Lac B (556 mg/100 g) was the highest level of any of the ingredients analysed; this high level was likely as a result of enrichment of MFGM type components, as described previously for PLs. The low cholesterol level in Lac A (42.2 mg/100 g) corresponded with the low PL and gangliosides levels, and was likely due the low fat content of the material.

TABLE 1
Mean and cholesterol contents of skimmed milk powder (SMP), whey
protein concentrate (WPC) and α--lactalbumin enriched WPC (Lac)
powders. Analysed values are presented as mean ± relative
standard deviation of data; sample size (n) is indicated in parentheses.

| | Cholesterol |
|---|---|
| Material | mg/100 g |
| SMP A (n = 5) | 24.2 ± 18.0% |
| SMP B (n = 5) | 23.1 ± 16.4% |
| SMP C (n = 5) | 20.7 ± 18.1% |
| SMP D (n = 5) | 21.9 ± 20.3% |
| WPC A (n = 5) | 77.0 ± 7.2% |
| WPC B (n = 5) | 85.3 ± 11.5% |
| Lac A (n = 2) | 42.2 |
| Lac B (n = 5) | 556 ± 5.1% |

*

3.2.3 Cholesterol Content of Infant Formula (IF) and Growing-Up-Milk (GUM)

Cholesterol levels were higher in IF 1 and GUM 1 than IF 2 and GUM 2, respectively (Table 2), in line with the presence of Lac B. Despite the absence of Lac B in IF 2, it still contained a reasonably high level of cholesterol; this was due to the presence of anhydrous milk fat in the formulation. GUM 2, which did not contain Lac B or anhydrous milk fat, had the lowest cholesterol content.

The cholesterol level in IF 1 (10 mg/100 mL) was similar to reported human milk levels; for example, Beggio et al. (2018) found mature human milk to have a mean cholesterol content of 11.3±3.0 mg/100 g. To the authors' knowledge, this study is the first infant formula with reported cholesterol levels similar to those of human milk, with commercial formulations typically containing ˜5 mg/100 mL (Claumarchirant et al., 2015). The higher cholesterol intake of breastfed infants is associated with several benefits, including enhanced modulation of cholesterol metabolism in infancy, and reduced plasma low-density lipoprotein levels (and associated reduced risk of cardiovascular disease) in adulthood (Delplanque, Gibson, Koletzko, Lapillonne & Strandvik, 2015). Supplying similar amounts of cholesterol to formula fed infants may potentially result in similar benefits.

TABLE 2
Mean levels of cholesterol in infant formula prototypes produced
using either Lac B (IF 1) or Lac A (IF 2), and growing-up
milk prototypes produced with a combination of WPC A and Lac
B (GUM 1) or only WPC A (GUM 2). Analysed values are presented
as mean ± relative standard deviation of data; sample
size (n) is indicated in parentheses. Nutrient levels per
100 mL are based on the reconstitution instructions for the
product: 12.9 g/100 mL for IF and 16.9 g/100 mL for GUM.

| | |
|---|---|
| Nutritional Powder Product | Cholesterol |

| Stage | mg/100 g | mg/100 mL |
|---|---|---|
| IF 1 (n = 5) | 77.4 ± 2.86% | 10.0 ± 2.86% |
| IF 2 (n = 5) | 41.6 ± 4.85% | 5.37 ± 4.85% |
| GUM 1 (n = 5) | 20.8 ± 4.5% | 3.51 ± 4.5% |
| GUM 2 (n = 3) | 15.9 ± 3.6% | 2.68 ± 3.6% |

1. A method for producing an α-lactalbumin enriched whey protein extract as a source of cholesterol in a synthetic nutritional composition wherein, the α-lactalbumin enriched (WPE) is obtained by a process comprising:
a. acidifying a whey protein product to pH 4 or below
b. forming a low calcium whey protein product by concentrating the proteins in the acidified whey protein until the calcium to protein ratio is less than about 0.001 and,
c. precipitating α-lactalbumin from the low-calcium whey protein product, wherein said precipitating step includes the sub-steps of:
(I) diluting the low-calcium whey protein product,
(II) adjusting the pH of the diluted low-calcium whey protein product to between 4 and 5 to form a precipitate and soluble proteins, and
(III) Separating the precipitate proteins from the soluble proteins; and
the WPE is added to said composition in an effective amount sufficient to ensure that said synthetic nutritional composition has a final concentration of cholesterol found in human breast milk.

2. Method according to claim 1 wherein the cholesterol content in synthetic nutritional composition ranges from 90-150 mg/L.

3. Method according to claim 1 wherein the WPE is also used to optimise the concentration of α-lactalbumin in said synthetic nutritional composition for an infant or child, and wherein said WPE is added to said composition in an effective amount sufficient to provide α-lactalbumin in one of the ranges found in human breast milk.

4. A method for providing nutrition to an infant or child comprising administering an α-lactalbumin enriched whey protein extract as a source of cholesterol in a synthetic nutritional composition wherein, the α-lactalbumin enriched (WPE) is obtained by a process comprising:
a. acidifying a whey protein product to pH 4 or below
b. forming a low calcium whey protein product by concentrating the proteins in the acidified whey protein until the calcium to protein ratio is less than about 0.001 and,
c. precipitating α-lactalbumin from the low-calcium whey protein product, wherein said precipitating step includes the sub-steps of:
(I) diluting the low-calcium whey protein product,
(II) adjusting the pH of the diluted low-calcium whey protein product to between 4 and 5 to form a precipitate and soluble proteins, and
(III) Separating the precipitate proteins from the soluble proteins; and
the WPE is added to said composition in an effective amount sufficient to ensure that said synthetic nutritional composition has a final concentration of cholesterol found in human breast milk wherein the synthetic nutritional composition comprises the WPE in a concentration in a range of 0.8 to 8 g/L.

5. Method according to claim 4 wherein said composition is an infant formula comprising 4 to 5 g/L of WPE and wherein said composition is preferably formulated for an infant of 6 to 12 months of age.

6. Method according to claim 4 wherein said composition is an infant formula comprising 4 to 5 g/L of WPE and wherein said composition is formulated for a child of 13 to 36 months of age.

7. Method according to claim 6 wherein said composition is an infant formula comprising 0.5 to 1.5 g/L of WPE and wherein said composition is formulated for a child of 3 to 8 years of age.

8. Method according to claim 4 wherein the cholesterol concentration in said composition is at least 90 mg/L.

9. Method according to claim 4 wherein the synthetic nutritional composition for an infant or child is a composition for consumption by infants either alone or in combination with human breast milk and is preferably an infant formula or human breast milk fortifier.

10. Method as defined in claim 4 for use in a method to promote and/or optimise the growth and/or the development in an infant or child to whom it is administered.

11. Method as defined in claim 4 for use in a method to promote and/or optimise immune defences in an infant or child to whom it is administered.

12. Method as defined in claim 4 for use in a method to promote and/or optimise fatty acid metabolism in an infant or child to whom it is administered.

13. (canceled)
